(19) United States
(12) Patent Application Publication
(10) Pub. No.: US 2020/0330732 A1
(43) Pub. Date: Oct. 22, 2020

(54) CATHETER DRESSINGS, SYSTEMS AND METHODS OF USE

(71) Applicant: Robert E. Helm, Rye Beach, NH (US)

(72) Inventor: Robert E. Helm, Rye Beach, NH (US)

(21) Appl. No.: 16/852,161

(22) Filed: Apr. 17, 2020

Related U.S. Application Data
(60) Provisional application No. 62/835,944, filed on Apr. 18, 2019.

(51) Int. Cl. A61M 25/02

(52) U.S. Cl. CPC A61M 25/02; A61M 2025/0206; A61M 2025/0246; A61M 2205/0205; A61M 2025/0266

(57) ABSTRACT

Catheter dressing-related devices, systems, and methods are disclosed. The devices may be configured to maintain sterility and to stabilize an inserted catheter to decrease catheter complication and failure rates. The devices may form two circumferential seals around the catheter to partially define a sealed chamber surrounding the catheter.

### RELATED APPLICATIONS

This application claims priority to U.S. Provisional Application No. 62/835,944, filed on Apr. 18, 2019 and titled “Catheter Dressing System and Method of Use,” which is hereby incorporated by reference in its entirety.

### TECHNICAL FIELD

The present invention relates to methods and devices for circumferentially sealing and securing medical devices in order to decrease complication and failure rates. Devices include, but are not limited to, vascular access catheters or other devices inserted into the human body.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a top view of an embodiment of a catheter dressing installed over a catheter.

FIG. 1B is a rear view of the catheter dressing of FIG. 1A.

FIG. 2A is a side view of the catheter dressing of FIG. 1A in a ready state.

FIG. 2B is a side view of the catheter dressing of FIG. 1A in an installed state.

FIG. 3 is a top view of another embodiment of a catheter dressing installed over an integrated catheter.

FIG. 4A is a top view of another embodiment of a catheter dressing installed over a dual lumen catheter.

FIG. 4B is a side view of the catheter dressing of FIG. 4A.

FIG. 4C is a rear view of the catheter dressing of FIG. 4A.

FIG. 5A is a top view of another embodiment of a catheter dressing installed over a tri-lumen catheter.

FIG. 5B is a side view of the catheter dressing of FIG. 5A.

FIG. 5C is a rear view of the catheter dressing of FIG. 5A.

FIG. 6 is a top view of another embodiment of a catheter dressing.

FIG. 7A is a rear view of the catheter dressing of FIG. 6 in a closed state.

FIG. 7B is a side view of the catheter dressing of FIG. 6 in a closed state.

FIG. 8A is an end view of the catheter dressing of FIG. 6 in an open state.

FIG. 8B is a side view of the catheter dressing of FIG. 6 in an open state.

FIG. 9 is a top view of the catheter dressing of FIG. 6 installed over a catheter.

FIG. 10A is a top view of another embodiment of a catheter dressing in an open state.

FIG. 10B is a top view of the catheter dressing of FIG. 10A in a closed state.

FIG. 10C is a side view of the catheter dressing of FIG. 10A in a closed state.

FIG. 11 is a side view of an alternate embodiment of the catheter dressing of FIG. 10A in an open state.

FIG. 12A is a top view of another embodiment of a catheter dressing in a closed state.

FIG. 12B is a top view of the catheter dressing of FIG. 12A in an open state.

FIG. 13A is an illustration of a method step of use of a catheter dressing in a ready state.

FIG. 13B is an illustration of another method step of use of the catheter dressing of FIG. 13A in an installed state.

### DETAILED DESCRIPTION

Peripheral, central, and other (e.g., intra-osseous, umbilical, etc.) vascular access catheter insertion and care may be complex and highly variable processes involving gathering, setting up, and using multiple disparate pieces of equipment. This may lead to a highly variable and contaminated result. Deficiencies of vascular access catheter care may fall into two groups: (1) inadequate catheter protection, leading to internal and external contamination of the catheter, and (2) inadequate catheter securement and stabilization, leading to tissue injury and catheter damage/dislodgement.

In certain instances, vascular access technology and application technique may not allow simple, clinically reproducible, and durable sterile-sealing protection of the vascular access catheter. Because dressing technology may not seal and fully protect the catheter and its insertion site, catheter contamination commonly occurs. Past and existing attempts at placing and preserving a sterile catheter and catheter site using an “aseptic no touch” technique have generally failed and have significantly contributed to the high rate of vascular catheter access failure (e.g., 25-50% of peripheral IV catheters fail). Compounding the problem of universal contamination is that catheter securement and stabilization technology may be inadequate in certain circumstances, leading to the need for a set of “compensatory” measures such as dedicated securement devices and anti-microbial adjuncts—measures that may increase clinical complexity, discomfort, and cost. The cost and complexity of these measured is a strong deterrent to their use.

It is therefore desirable in certain instances to have a device that seals, stabilizes, and secures an inserted vascular access catheter—or any device inserted into the body—in a way that is simple and non-complex to apply with a minimal number of steps, effective in achieving intended therapeutic goals, and able to achieve this goal in a highly reproducible manner.

The device presented achieves these goals by providing for a fully sealed and secured vascular (or other body cavity) access device. The devices may be used to fully seal and secure any type of medical device that is inserted into the body of a patient. For example, the devices disclosed herein can be used to fully and seal straight peripheral intravenous (IV) catheters, integrated peripheral IV catheters, peripherally inserted central (PIC) catheters, midline catheters, central line catheters, hemodialysis catheters, umbilical catheters, IABPs, LVADs, ECMO machines, PEG feeding tubes, percutaneous nephrostomy devices, epidural pain catheters, temporary pacemaker access devices, etc. Central to achieving this in a simple, easy-to-use, and highly reproducible manner is that a dressing device has two basic configurations: open and closed. In the open state it can be easily placed over most catheter devices without disturbing the catheter. In the closed state, it forms two durable seals—one around the inserted catheter at a skin insertion site, and one around the catheter where it exits the closed dressing device. In the closed and locked-down state, the device is in a position to seal the catheter and insertion site against external contamination and to stabilize and secure the contained catheter. This application is directed at delineating mechanisms and methods for achieving this circumferential securing seal around an inserted device.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to a catheter dressing, the proximal end of the catheter dressing refers to the end nearest a catheter hub, and the distal end refers to the opposite end, the end nearest the catheter tube. Thus, if at one or more points in a procedure a practitioner changes the orientation of a catheter dressing, as used herein the term “proximal end” always refers to the catheter hub end of the catheter dressing (even if the distal end is temporarily closer to the practitioner).

“Fluid” is used in its broadest sense, to refer to any fluid, including both liquids and gases as well as solutions, compounds, suspensions, etc., which generally behave as fluids.

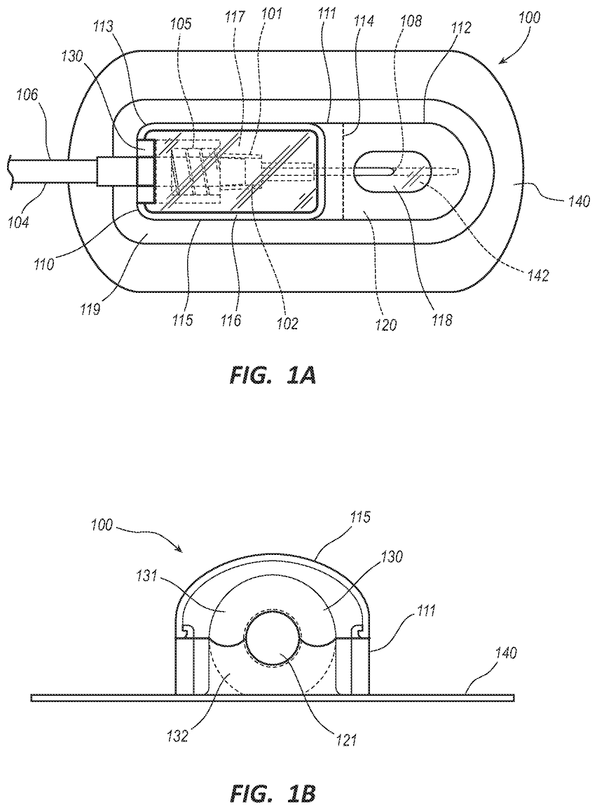

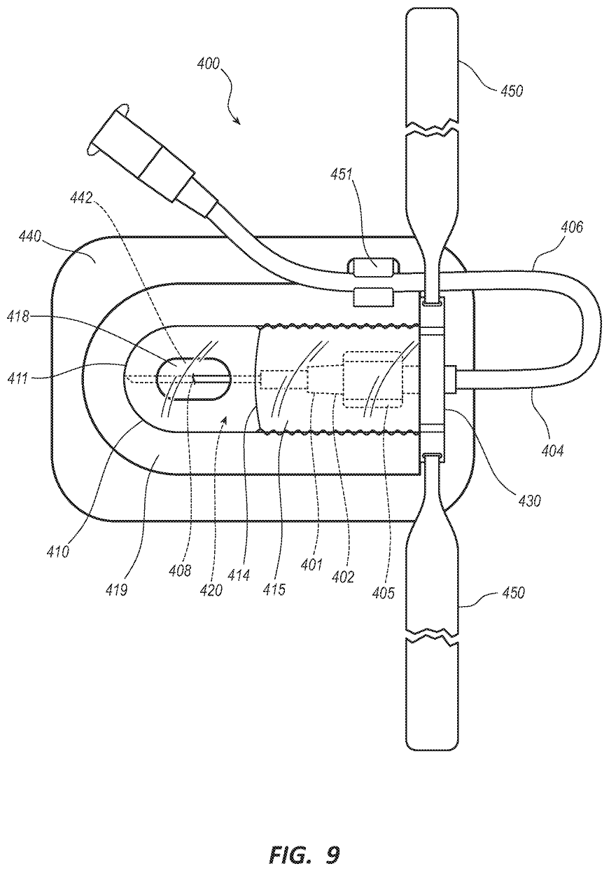

FIGS. 1-14 illustrate different views of several catheter dressings and related components. In certain views each catheter dressing may be coupled to, or shown with, additional components not included in every view. Further, in some views only selected components are illustrated, to provide detail into the relationship of the components. Some components may be shown in multiple views but not discussed in connection with every view. Disclosure provided in connection with any figure is relevant and applicable to disclosure provided in connection with any other figure or embodiment.

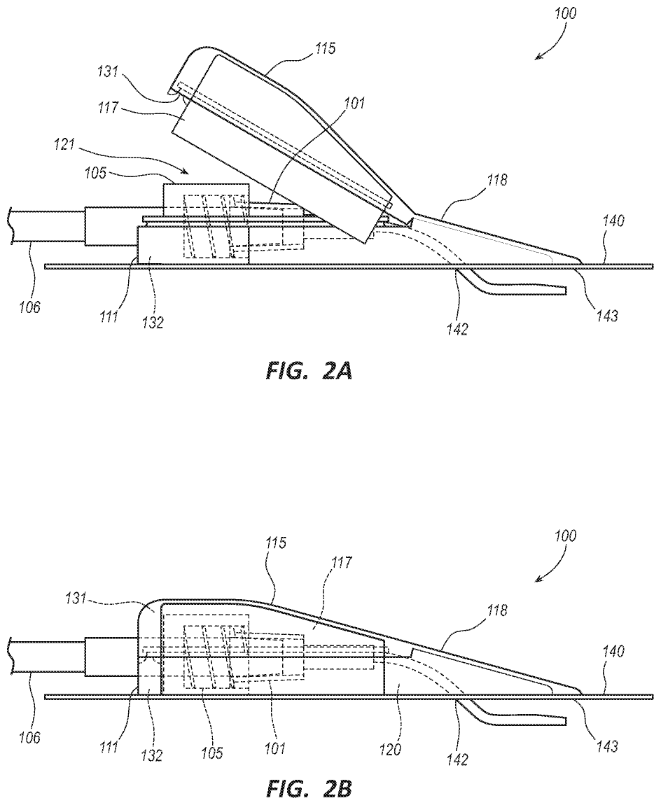

FIGS. 1A-2B depict an embodiment of a catheter dressing 100. In the illustrated embodiment, the catheter dressing 100 is composed of a housing 110 and a base plate 140. The housing 110 may be composed of a body 111, a displaceable member or lid 115, a sterile or sealed chamber 120, and a circumferential seal member 130.

In the illustrated embodiment, the body 111 includes a generally rectangular footprint. In other embodiments, the body 111 may be circular, oval, square, etc. A distal portion 112 of the body 111 is shown to taper upwardly toward a proximal portion 113. In another embodiment, the distal portion 112 may be level with the proximal portion 113. The sealed chamber 120 may be partially defined by an inside surface of the body 111. The body 111 may be formed of any suitable rigid or semi-rigid thermoplastic material. For example, the body 111 may be formed from polypropylene, polyethylene, polycarbonate, polyvinylchloride, etc., using any suitable forming technique, such as thermoforming, injection molding, etc. An optional transparent viewing window 118 may be disposed adjacent the distal portion 112 such that a catheter 101 and a catheter insertion site 108 may be viewed by a clinician. In another embodiment, the body 111 may be wholly transparent or translucent.

The displaceable member 115 is shown in the illustrated embodiment to be disposed adjacent the proximal portion 113 of the body 111. The displaceable member 115 may be rectangular in shape and sized to fit within an opening 121 disposed adjacent the proximal portion 113. The displaceable member 115 may be formed of a material similar to the material forming the body 111 and may be integral with the body 111. In other embodiments, the displaceable member 115 may be a separate component from the body 111. A gasket 116 may be coupled to a periphery of the displaceable member 115. The gasket 116 may be formed of a compressible material, such as silicone rubber, silicone foam, polyurethane foam, thermoplastic elastomer, etc. The displaceable member 115 can be configured to engage with the body 111 to seal the opening 121 when the displaceable member 115 is in a closed state. Sealing of the opening 121 by the displaceable member 115 when in the closed state may prevent microbial contamination of the catheter 101. The displaceable member 115 may engage with the body 111 with a snap fit, a friction fit, etc. The engagement of the displaceable member 115 to the body 111 may be fixed such that the displaceable member 115 cannot be displaced from the body 111. In another embodiment, the engagement of the displaceable member 115 may be releasable such that the displaceable member 115 can be selectively displaced from the body 111.

In the depicted embodiment, a hinge 114 is shown to couple the displaceable member 115 to the body 111. The hinge 114 may be a flexible hinge that allows for multiple actuations and provides a sealed coupling between the displaceable member 115 and the body 111. For example, the hinge 114 may be a thinned wall, a living hinge, an H-style hinge, a super hinge, etc. The hinge 114 may be integral with the body 111 and/or the displaceable member 115. In other embodiments, the hinge 114 may be a separate component that is coupled to the body 111 and the displaceable member 115 using any suitable technique, such as gluing, welding, over molding, etc.

The circumferential seal member 130 is shown in the figures disposed adjacent a proximal end of the body 111. The circumferential seal member 130 may comprise a first or upper seal member 131 and a lower or second seal member 132. The seal members 131, 132 may be generally cylindrical “C” shaped. The upper seal member 131 may include a male portion configured to couple with a female portion of the lower seal member 132 to form a seal between the seal members 131, 132. The upper seal member 131 may be coupled to the displaceable member 115, and the lower seal member 132 may be coupled to the body 111. This configuration allows the upper seal member 131 to be displaced away from the lower seal member 132 when the displaceable member 115 is displaced away from the body 111, and to be coupled to the lower seal member 132 when the displaceable member 115 is engaged with the body 111. The size of the opening 121 may be selectively increased when the displaceable member 115 is displaced away from the body 111. The increased size of the opening 121 may permit passage of at least a portion of an extension tube 106 when the catheter dressing 100 is installed onto the patient. The displaceable member 115 may be displaced toward the body 111, resulting in closure of the opening 121 and coupling of the upper seal member 131 with the lower seal member 132. When coupled, the seal members 131, 132 may form a second circumferential seal around the catheter or extension tube 106. The second circumferential seal may surround the catheter 101 and/or extension tube 106 a full 360 degrees. In other words, the first circumferential seal may surround the catheter 101 and/or extension tube 106 without breaks in the second circumferential seal. The second circumferential seal may be configured to prevent microbial contamination of the catheter 101 and the insertion site 108. The second circumferential seal may also be configured to stabilize the catheter 101 from longitudinal movement. The seal members 131, 132 may be formed from a compressible material, such as silicone rubber, silicone foam, polyurethane foam, thermoplastic elastomer, etc.

As depicted in the illustrated embodiment, the base plate 140 is coupled to the housing 110. The housing 110 may include a flange 119 to facilitate coupling of the base plate 140 to the housing 110 using any suitable technique, such as welding, gluing, bonding, etc. The base plate 140 may have a generally rectangular shape with radiused or rounded corners. In other embodiments, the base plate 140 may be of any suitable shape, such as circular, oval, square, etc. The base plate 140 may be formed of a single layer of material. In other embodiments, the base plate 140 may be formed of two, three, four, or more layers of the same material or of different materials. The material of the base plate 140 may be any suitable material that is non-irritating to the patient's skin and is conformable to a contour of the portion of the patient's body where the catheter dressing 100 is installed. The material of the base plate 140 may be a hydrocolloid, a polyurethane film, etc. The material of the base plate 140 may be self-adhering to the patient's skin or it may include an adhesive to facilitate adhesion of the base plate 140 to the patient's skin. The material of the base plate 140 may be configured to permit moisture vapor transmission. The base plate 140 may include an antimicrobial agent disposed within or on a surface of the base plate 140. The antimicrobial agent may prevent microbial contamination of the catheter 101 when the catheter dressing 100 is in an installed state. A release liner can be releasably coupled to a bottom surface of the base plate 140 to protect the surface until the base plate 140 is coupled to the patient's skin. The release liner may be removed laterally or longitudinally.

In the depicted embodiment, the base plate 140 includes an aperture 142 disposed in a proximal portion 143 of the base plate 140. The aperture 142 may be sized to permit passage of the base plate 140 over an inserted catheter 101 and the extension tube 106 when the catheter dressing 100 is installed onto the patient. When installed, the base plate 140 may surround the catheter 101 at the insertion site 108 such that a first circumferential seal is formed around the catheter 101. The first circumferential seal may be in the plane of the base plate 140 and surround the catheter 101 a full 360 degrees. In other words, the first circumferential seal may surround the catheter 101 without breaks in the first circumferential seal such that microbial contamination of the catheter 101 and insertion site 108 may be prevented.

In the depicted embodiment, the sterile or sealed chamber 120 may be at least partially defined by the body 111, the displaceable member 115, the base plate 140, and the circumferential seal member 130. When the catheter dressing 100 is installed, the sealed chamber 120 may be configured to provide a substantially sterile environment around the catheter 101 and insertion site 108. In other words, the environment of the sealed chamber 120 may be substantially free of microorganisms.

The catheter dressing 100 may be used to stabilize and maintain sterility of the catheter 101. In certain instances, stabilization and sterility of the catheter 101 may reduce failure of the catheter 101 prior to completion of therapy. Catheter failure may require removal of the catheter 101 and replacement with another catheter 101 prior to completion of treatment of the patient. Stabilization and sterility of the catheter 101 may also reduce a risk of catheter-related infection.

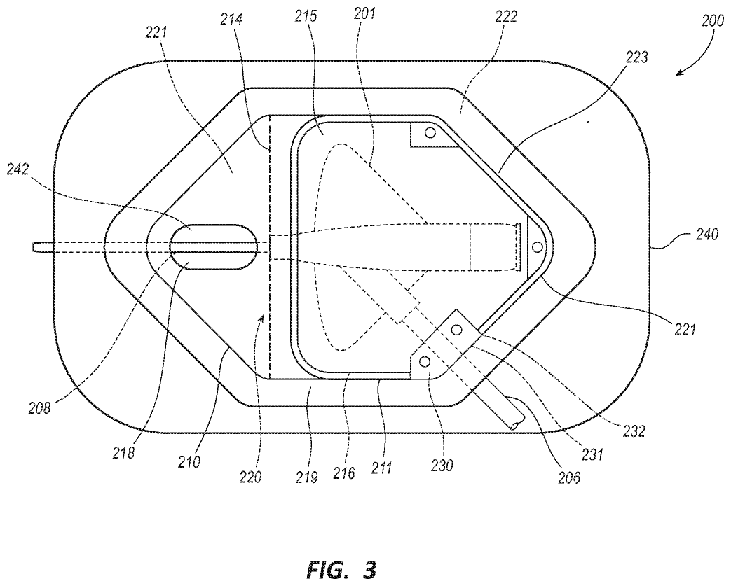

FIG. 3 depicts an embodiment of a catheter dressing 200 that resembles the catheter dressing 100 described above in certain respects. Accordingly, like features are designated with like reference numerals, with the leading digit incremented to “2.” For example, the embodiment depicted in FIG. 3 includes a housing 210 that may, in some respects, resemble the housing 110 of FIG. 1A. Relevant disclosure set forth above regarding similarly identified features thus may not be repeated hereafter. Moreover, specific features of the catheter dressing 100 and related components shown in FIGS. 1A-2B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the catheter dressing 200 and related components depicted in FIG. 3. Any suitable combination of the features, and variations of the same, described with respect to the catheter dressing 100 and related components illustrated in FIGS. 1A-2B can be employed with the catheter dressing 200 and related components of FIG. 3, and vice versa. This pattern of disclosure applies equally to further embodiments depicted in subsequent figures and described hereafter, wherein the leading digits may be further incremented.

FIG. 3 depicts another embodiment of a catheter dressing 200. In the illustrated embodiment, the catheter dressing 200 is composed of a housing 210 and a base plate 240. The housing 210 may be composed of a body 211, a displaceable member or lid 215, a sterile or sealed chamber 220, and a circumferential seal member 230.

In the illustrated embodiment, the body 211 includes two angled distal sides, two lateral sides, and two angled proximal sides 223. In other embodiments, the body 211 may include a distal side, two lateral sides, and two angled proximal sides 223. The proximal sides 223 may be angled relative to a longitudinal axis of the body 211 such that they form a proximal point at a junction of the proximal sides 223. The proximal sides 223 may be perpendicular to a longitudinal axis of an integrated extension tube 206 of a catheter 201. The sealed chamber 220 may be partially defined by an inside surface of the body 211. An optional transparent viewing window 218 may be disposed adjacent the distal end of the body 211, such that the catheter 201 and a catheter insertion site 208 may be viewed by a clinician.

In the illustrated embodiment, the displaceable member 215 may be similarly shaped to the body 211 and sized to fit within an opening 221. A gasket 216 may be coupled to a periphery of the displaceable member 215. The displaceable member 215 can be configured to engage with the body 211 to seal the opening 221 when the displaceable member 215 is in a closed state. For example, in the depicted embodiment, the displaceable member 215 is engaged with the housing 210 at engagement members 222. The engagement members 222 may be configured to provide a snap fit, friction fit, or other suitable type of engagement. The engagement of the displaceable member 215 to the body 211 may be permanent such that the displaceable member 115 cannot be displaced from the body 211. In another embodiment, the engagement of the displaceable member 215 may be releasable such that the displaceable member 215 can be selectively displaced from the body 211.

In the depicted embodiment, a hinge 214 is shown to couple the displaceable member 215 to the body 211. The hinge 214 may be a flexible hinge that allows for multiple actuations and provides a sealed coupling between the displaceable member 215 and the body 211. The hinge 214 may be integral with the body 211 and/or the displaceable member 215. In other embodiments, the hinge 214 may be a separate component that is coupled to the body 211 and displaceable member 215 using any suitable technique, such as gluing, welding, over molding, etc. The illustrated embodiment depicts the hinge 214 being oriented perpendicular to a longitudinal axis of the body 211 such that the displaceable member 215 may be displaced from a distal end. In other embodiments, the hinge 214 may be oriented parallel to the longitudinal axis of the body 211 such that the displaceable member 215 may be displaced from a lateral side of the body 211.

In the illustrated embodiment, the circumferential seal member 230 is shown disposed adjacent a proximal side 223 of the body 211. The circumferential seal member 230 may comprise a first or upper seal member 231 and a second or lower seal member 232. The upper seal member 231 may be coupled to the displaceable member 215, and the lower seal member 232 may be coupled to the body 211. This configuration allows the upper seal member 231 to be displaced away from the lower seal member 232 when the displaceable member 215 is displaced away from the body 211 in an open state and to be coupled to the lower seal member 232 when the displaceable member 215 is engaged with the body 211 in the closed state. The size of the opening 221 may be selectively increased when the displaceable member 215 is in the open state. The increased size of the opening 221 may permit passage of the catheter 201 and an extension tube 206 when the catheter dressing 200 is installed onto the patient. The displaceable member 215 may be displaced toward the body 211 to a closed state resulting in closure of the opening 221 and coupling of the upper seal member 231 with the lower seal member 232. When coupled, the seal members 231, 232 may form a second circumferential seal around the catheter 201 or extension tube 206.

As depicted in the illustrated embodiment, the base plate 240 is coupled to the housing 210. The housing 210 may include a flange 219 to facilitate coupling of the base plate 240 to the housing 210. The base plate 240 may have a generally rectangular shape with radiused or rounded corners. The base plate 240 may be formed of a single layer of material. The material of the base plate 240 may be any suitable material that is non-irritating to the patient's skin and is conformable to a contour of the portion of the patient's body where the catheter dressing 200 is installed. The base plate 240 may include an antimicrobial agent disposed within or on a surface of the base plate 240.

In the depicted embodiment, the base plate 240 includes an aperture 242. The aperture 242 may be sized to permit passage of the base plate 240 over the inserted catheter 201 and extension tube 206 when the catheter dressing 200 is installed onto the patient. When installed, the base plate 240 may surround the catheter 201 at the insertion site 208 such that a first circumferential seal is formed around the catheter 201.

In the depicted embodiment, the sterile or sealed chamber 220 may be at least partially defined by the body 211, the displaceable member 215, the base plate 240, and the circumferential seal member 230. When the catheter dressing 200 is installed, the sealed chamber 220 may be configured to provide a substantially sterile environment around the catheter 201 and insertion site 208. In other words, the environment of the sealed chamber 220 may be substantially free of microorganisms.

The catheter dressing 200 may be used to stabilize and maintain sterility of the catheter 201 having an integrated extension tube 206. In certain instances, stabilization and sterility of the catheter 201 may reduce failure of the catheter 201 prior to completion of therapeutic therapy. Catheter failure may require removal of the catheter 201 and replacement with another catheter 201. Stabilization and sterility of the catheter 201 may also reduce a risk of catheter-related infection.

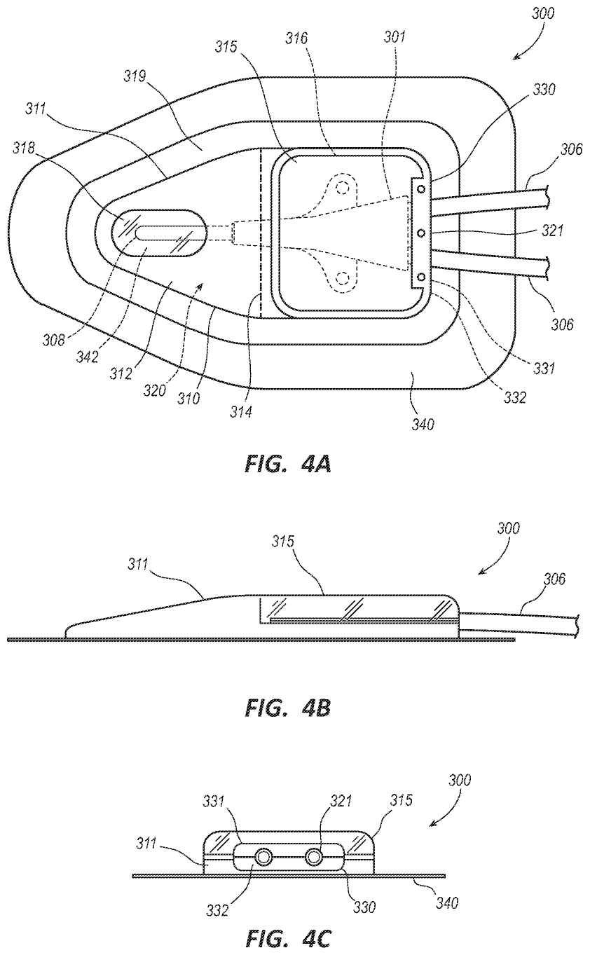

FIGS. 4A-4C depict another embodiment of a catheter dressing 300. In the illustrated embodiment, the catheter dressing 300 is composed of a housing 310 and a base plate 340. The housing 310 may be composed of a body 311, a displaceable member or lid 315, a sterile or sealed chamber 320, and a circumferential seal member 330.

In the illustrated embodiment, the body 311 includes a generally rectangular footprint. The sealed chamber 320 may be partially defined by an inside surface of the body 311. The body 311 may be formed of any suitable rigid or semi-rigid thermoplastic material. An optional transparent viewing window 318 may be disposed adjacent a distal portion 312 such that a catheter 301 and catheter insertion site 308 may be viewed by a clinician. In another embodiment, the body 311 may be wholly transparent or translucent.

In the illustrated embodiment, the displaceable member 315 may include a shape similar to the shape of the body 311 and sized to fit within an opening 321. A gasket 316 may be coupled to a periphery of the displaceable member 315. The displaceable member 315 can be configured to engage with the body 311 to seal the opening 321 when the displaceable member 315 is in a closed state. The engagement of the displaceable member 315 to the body 311 may be permanent such that the displaceable member 315 cannot be displaced from the body 311. In another embodiment, the engagement of the displaceable member 315 may be releasable such that the displaceable member 315 can be selectively displaced from the body 311.

In the depicted embodiment, a hinge 314 is shown to couple the displaceable member 315 to the body 311. The hinge 314 may be a flexible hinge that allows for multiple actuations and provides a sealed coupling between the displaceable member 315 and the body 311. The hinge 314 may be integral with the body 311 and/or the displaceable member 315. In other embodiments, the hinge 314 may be a separate component that is coupled to the body 311 and displaceable member 315 using any suitable technique, such as gluing, welding, over molding, etc. The illustrated embodiment depicts the hinge 314 being oriented perpendicular to a longitudinal axis of the body 311 such that the displaceable member 315 may be displaced from a distal end. In other embodiments, the hinge 314 may be oriented parallel to the longitudinal axis of the body 311 such that the displaceable member 315 may be displaced from a lateral side of the body 311.

The illustrated embodiment depicts the circumferential seal member 330 configured to receive two extension tubes 306 and disposed adjacent a proximal end of the body 311. In other embodiments, the catheter dressing 300 may comprise three, four, five, or more circumferential seal members 330. Each of the circumferential seal members 330 may comprise a first or upper seal member 331 and a second or lower seal member 332. The upper seal members 331 may be coupled to the displaceable member 315, and the lower seal members 332 may be coupled to the body 311. This configuration allows the upper seal members 331 to be displaced away from the lower seal members 332 when the displaceable member 315 is displaced away from the body 311 in an open state and to be coupled to the lower seal members 332 when the displaceable member 315 is engaged with the body 311 in the closed state. The size of the opening 321 may be selectively increased when the displaceable member 315 is in the open state. The increased size of the opening 321 may permit passage of one, two, three, or more extension tubes 306 when the catheter dressing 300 is installed onto the patient. The displaceable member 315 may be displaced toward the body 311 to a closed state resulting in closure of the opening 321 and coupling of the upper seal members 331 with the lower seal members 332. When coupled, the seal members 331, 332 may form a second circumferential seal around the catheter 301 or extension tubes 306.

As depicted in the illustrated embodiment of FIGS. 4A-4C, the base plate 340 is coupled to the housing 310. The housing 310 may include a flange 319 to facilitate coupling of the base plate 340 to the housing 310. The base plate 340 may have a generally rectangular shape with radiused or rounded corners. The base plate 340 may be formed of a single layer of material. The material of the base plate 340 may be any suitable material that is non-irritating to the patient's skin and is conformable to a contour of the portion of the patient's body where the catheter dressing 300 is installed. The base plate 340 may include an antimicrobial agent disposed within or on a surface of the base plate 340.

In the depicted embodiment, the base plate 340 includes an aperture 342. The aperture 342 may be sized to permit passage of the base plate 340 over the inserted catheter 301 and extension tubes 306 when the catheter dressing 300 is installed onto the patient. When installed, the base plate 340 may surround the catheter 301 at the insertion site 308 such that a first circumferential seal is formed around the catheter 301.

In the depicted embodiment, the sterile or sealed chamber 320 may be at least partially defined by the body 311, the displaceable member 315, the base plate 340, and the circumferential seal member 330. When the catheter dressing 300 is installed, the sealed chamber 320 may be configured to provide a substantially sterile environment around the catheter 301 and insertion site 308. In other words, the environment of the sealed chamber 320 may be substantially free of microorganisms.

The catheter dressing 300 may be used to stabilize and maintain sterility of the catheter 301 having a plurality of extension tubes 306. For example, the catheter dressing 300 may be used for multi-lumen PIC catheters, midline catheters, central venous catheters, etc. In certain instances, stabilization and sterility of the catheter 301 may reduce failure of the catheter 301 prior to completion of therapeutic therapy. Failure of the catheter 301 may require removal of the catheter 301 and replacement with another catheter 301. Stabilization and sterility of the catheter 301 may also reduce a risk of catheter-related infection.

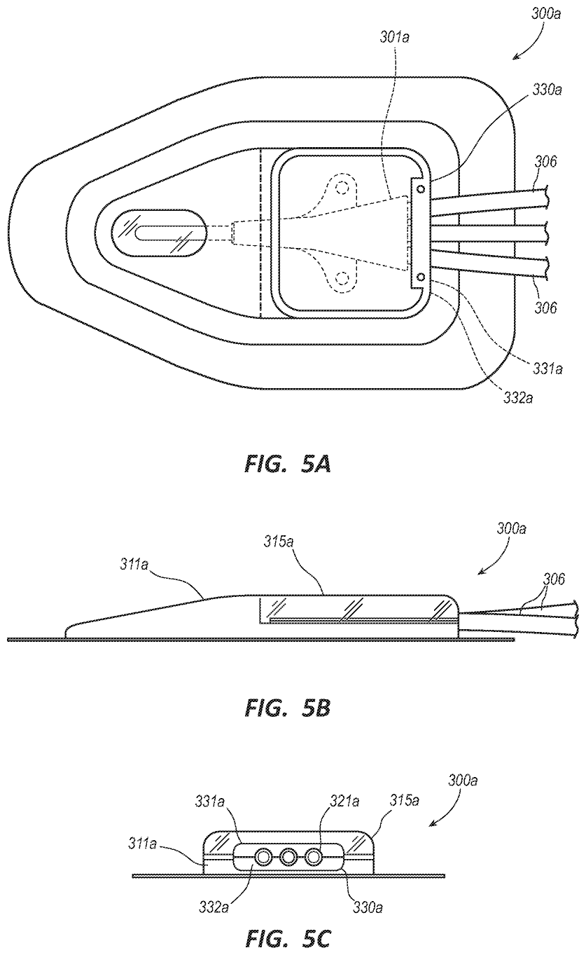

FIGS. 5A-5C depict an embodiment of a catheter dressing 300a. The catheter dressing 300a is configured to be installed over a multi-lumen catheter 301a having three integrated extension tubes 306 (e.g., a PIC catheter or central venous catheter). A circumferential seal member 330a may comprise an upper seal member 331a coupled to a displaceable member 315a and a lower seal member 332a coupled to a body 311a. The seal members 331a, 332a may be configured to define three openings 321a when the displaceable member 315a is in a closed state.

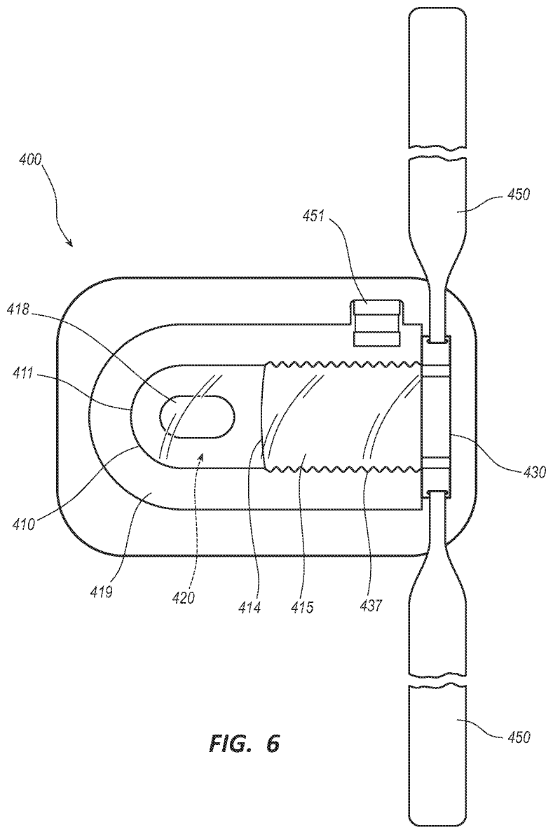

FIGS. 6-9 depict another embodiment of a catheter dressing 400. In the illustrated embodiment, the catheter dressing 400 is composed of a housing 410 and a base plate 440. The housing 410 may be composed of a body 411, a displaceable member 415, a sealed chamber 420, and a circumferential seal member 430.

In the illustrated embodiment, the body 411 includes a generally rectangular footprint. A sterile or sealed chamber 420 may be partially defined by an inside surface of the body 411. The body 411 may be formed of any suitable rigid or semi-rigid thermoplastic material. An optional transparent viewing window 418 may be disposed adjacent a distal portion of the body 411 such that a catheter 401 and catheter insertion site 408 may be viewed by a clinician. In another embodiment, the body 411 may be wholly transparent or translucent.

The displaceable member 415 is shown in the illustrated embodiment to be disposed adjacent a proximal portion of the body 411. The displaceable member 415 may be rectangular in shape. The displaceable member 415 may be formed of a material similar to the material forming the body 411 and may be integral with the body 411. In other embodiments, the displaceable member 415 may be a separate component from the body 411.

In the depicted embodiment, a hinge 414 is shown to couple the displaceable member 415 to the body 411. The hinge 414 may be a flexible hinge that allows for multiple actuations and provides a sealed coupling between the displaceable member 415 and the body 411. For example, the hinge 414 may be a thinned wall, a living hinge, an H-style hinge, a super hinge, etc. The hinge 414 may be integral with the body 411 and/or the displaceable member 415. In other embodiments, the hinge 414 may be a separate component that is coupled to the body 411 and displaceable member 415 using any suitable technique, such as gluing, welding, over molding, etc.

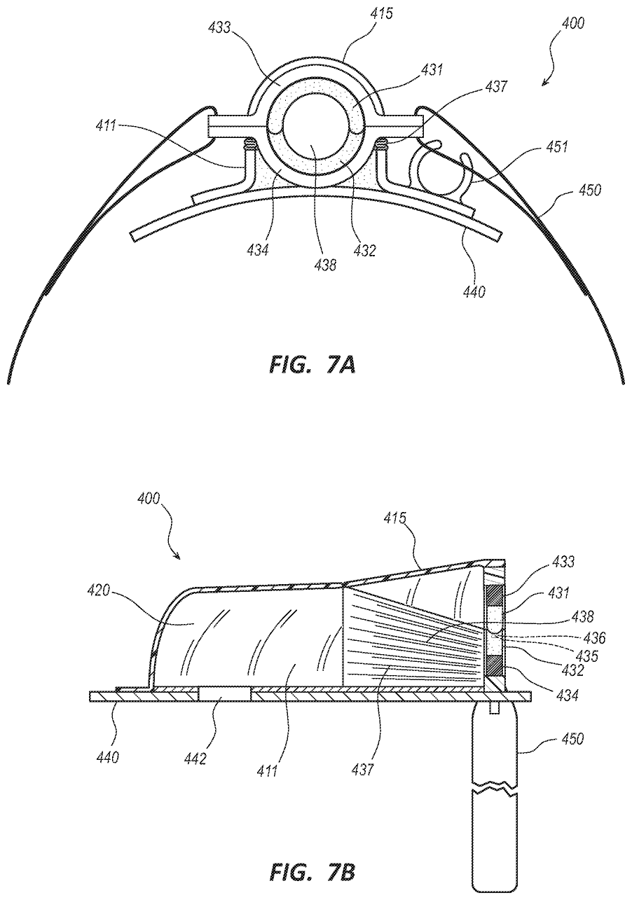

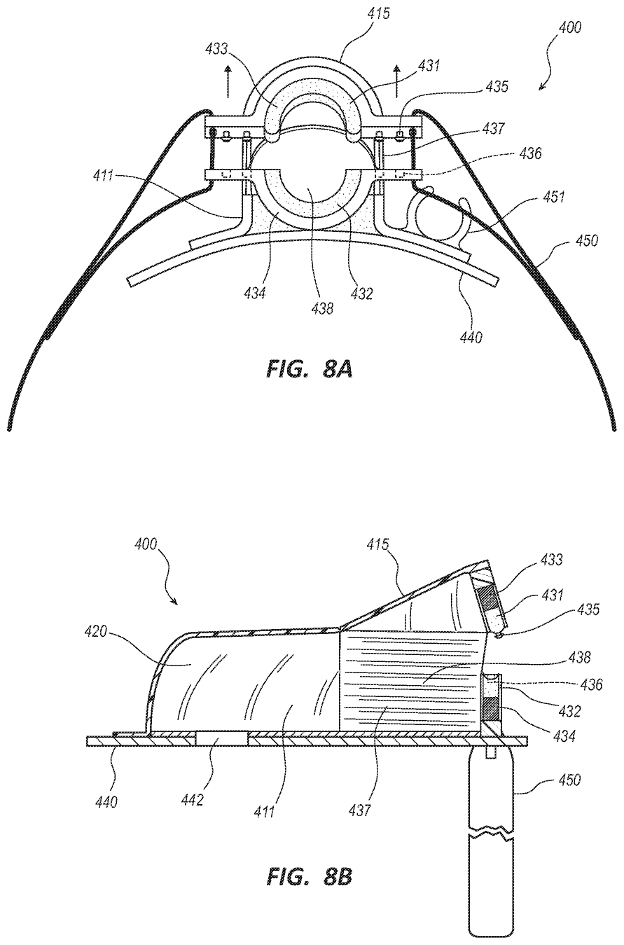

The circumferential seal member 430 is shown disposed adjacent a proximal end of the body 411. The circumferential seal member 430 may comprise a first or upper seal member 431, a first or upper support 433, a second or lower seal member 432, and a second or lower support 434. The upper and lower seal members 431, 432 may be coupled to the upper and lower supports 433, 434, respectively. The upper support 433 may be coupled to the displaceable member 415, and the lower support 434 may be coupled to the body 411. This configuration allows the upper seal member 431 and the upper support 433 to be displaced away from the lower seal member 432 and the lower support 434 when the displaceable member 415 is displaced away from the body 411 in an open state, as shown in FIGS. 8A-8B. When the displaceable member 415 is displaced toward the body 411, the upper seal member 431 may be coupled to the lower seal member 432, and the upper support 433 may be coupled to the lower support 434 in a closed state, as shown in FIGS. 7A-7B.

The upper support 433 may be coupled to the lower support 434 using any suitable coupling technique. For example, in the illustrated embodiment as depicted in FIG. 8A, the upper support 433 may comprise at least one mushroom head post 435 disposed on at least one side of the upper support 433. The lower support 434 may comprise at least one post receiver 436 disposed on at least one side of the lower support 434. The post 435 may be aligned opposite the post receiver 436 such that when the upper support 433 is coupled to the lower support 434, the post 435 is received by the post receiver 436. In certain embodiments, the upper support 433 may be coupled to the lower support 434 using techniques such as adhesive, static magnets, clips, etc. In some embodiments, the upper support 433 may be un-releasably coupled to the lower support 434. In other embodiments, the upper support 433 may be releasably coupled to the lower support 434.

When coupled, the seal members 431, 432 may form a second circumferential seal around the catheter 401 or an extension tube 406. The second circumferential seal may surround the catheter 401 and/or extension tube 406 a full 360 degrees. In other words, the first circumferential seal may surround the catheter 401 and/or extension tube 406 without breaks in the second circumferential seal. The second circumferential seal may be configured to prevent microbial contamination of the catheter 401 and the insertion site 408. The second circumferential seal may also be configured to stabilize the catheter 401 from longitudinal movement. The seal members 431, 432 may be formed from a compressible material, such as silicone rubber, silicone foam, polyurethane foam, thermoplastic elastomer, etc.

A stretchable membrane 437 may be coupled to the upper support 433, the lower support 434, the displaceable member 415, the body 411, and the base plate 440. The membrane 437 may be configured to form a channel 438 for passage of the extension tube 406 when the catheter dressing 400 is installed onto a patient. When the circumferential seal member 430 is in the open state, the membrane 437 is stretched between the upper and lower supports 433, 434, increasing a size of the channel 438 to facilitate passage of the extension tube 406. When the circumferential seal member 430 is in the closed or sealing state, the membrane 437 is not stretched and the size of the channel 438 is smaller such that the membrane 437 surrounds the catheter 401. The membrane 437 may be formed from any suitable elastomeric material, such as silicone, thermoplastic elastomer, rubber, etc.

As depicted in the illustrated embodiment, the base plate 440 is coupled to the housing 410. The housing 410 may include a flange 419 configured to facilitate coupling of the base plate 440 to the housing 410 using any suitable technique, such as welding, gluing, bonding, etc. The base plate 440 may have a generally rectangular shape with radiused or rounded corners. The base plate 440 may be formed of a single layer of material. In other embodiments, the base plate 440 may be formed of two, three, four, or more layers of the same material or of different materials. The material of the base plate 440 may be any suitable material that is non-irritating to the patient's skin and is conformable to a contour of the portion of the patient's body where the catheter dressing 400 is installed. The base plate 440 may include an antimicrobial agent disposed within or on a surface of the base plate 440. The antimicrobial agent may prevent microbial contamination of the catheter 401 when the catheter dressing 400 is in an installed state.

In some embodiments, the catheter dressing 400 may include a stabilization strap or band 450. The strap 450 may include a self-adhering material, such as a hook-and-loop material or adhesive. In other embodiments, the strap 450 may include a coupling mechanism, such as a buckle, snaps, posts and holes, etc. As depicted in the illustrated embodiment, the strap 450 may comprise a first strap portion coupled to a first side of the circumferential seal member 430, and a second strap portion coupled to a second side of the circumferential seal member 430. In another embodiment, the first strap portion may be coupled to a first side of the body 411, and the second strap portion may be coupled to a second side of the body 411. The strap 450 may be configured to be wrapped around a portion of the patient (e.g., a leg or arm) to stabilize the catheter dressing 400 when installed on the patient. The strap 450 may stabilize the catheter dressing 400 from longitudinal and/or lateral movement.

In the depicted embodiment, the catheter dressing 400 may include a tube clip 451 coupled to a lateral portion of the body 411. The tube clip 451 may be configured to receive and retain the extension tube 406 following installation of the catheter dressing 400. The extension tube 406 may extend proximally from the catheter dressing 400 and then be looped distally to be received by the tube clip 451. The loop of the extension tube 406 helps to prevent proximal tension on the catheter 401 that may result in dislodgement of the catheter 401.

In the depicted embodiment, the base plate 440 may include an aperture 442 disposed in a proximal portion of the base plate 440. The aperture 442 may be sized to permit passage of the base plate 440 over the inserted catheter 401 and the extension tube 406 when the catheter dressing 400 is installed onto the patient. When installed, the base plate 440 may surround the catheter 401 at the insertion site 408 such that a first circumferential seal is formed around the catheter 401. The first circumferential seal may be in the plane of the base plate 440 and surround the catheter 401 a full 360 degrees. In other words, the first circumferential seal may surround the catheter 401 without breaks in the first circumferential seal such that microbial contamination of the catheter 401 and insertion site 408 may be prevented.

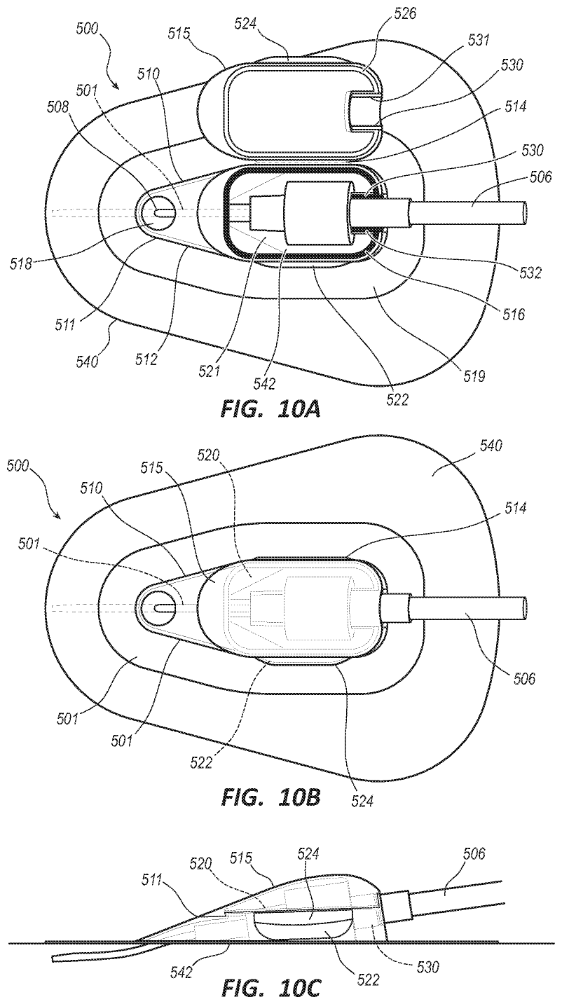

FIGS. 10A-10C depict an embodiment of a catheter dressing 500. In the illustrated embodiment, the catheter dressing 500 is composed of a housing 510 and a base plate 540. The housing 510 may be composed of a body 511, a displaceable member or lid 515, and a circumferential seal member 530.

In the illustrated embodiment, the body 511 includes a generally rectangular footprint having a narrowing distal portion 512. The body 511 may be formed of any suitable rigid or semi-rigid thermoplastic material. For example, the body 511 may be formed from polypropylene, polyethylene, polycarbonate, polyvinylchloride, etc., using any suitable forming technique, such as thermoforming, injection molding, etc. An optional transparent viewing window 518 may be disposed adjacent the distal portion 512 such that a catheter 501 and catheter insertion site 508 may be viewed by a clinician.

In the depicted embodiment, the displaceable member 515 is configured to be displaced from an open state, as shown in FIG. 10A, to a closed state as shown in FIGS. 10B and 10C. The displaceable member 515 may be sized to cover and seal an opening 521 of the body 511 when the displaceable member 515 is in the closed state. The displaceable member 515 can be laterally displaced away from the opening 521 to permit access to an interior of the body 511 through the opening 521 when the displaceable member 515 is in the open state. A hinge 514 is shown to couple the displaceable member 515 to the body 511. The hinge 514 is disposed on a lateral portion of the body 511 such that the displaceable member 515 is displaced laterally relative to a longitudinal axis of the body 511. The hinge 514 may be a flexible hinge that allows for multiple actuations and provides a sealed coupling between the displaceable member 515 and the body 511.

The body 511 can include a first engagement or clasp member 522 disposed on a lateral portion opposite of the hinge 514. The first engagement member 522 may be configured to matingly engage with a second engagement member 524 and securely couple the displaceable member 515 to the body 511 when the displaceable member 515 is in the closed state. The engagement members 522, 524 may include any suitable configuration capable of securing the displaceable member 515 and the body 511 together. For example, the engagement members 522, 524 may be frictional or snap fit joints, such as cantilever, annular, etc. In some embodiments, the engagement members 522, 524 can be fixedly coupled when the displaceable member 515 is in the closed state. In other embodiments, the engagement members 522, 524 can be releasably coupled when the displaceable member 515 is in the closed state. When in the closed state, the engagement members 522, 524 are configured to apply a compressive force to a first seal member 531 and a second seam member 532 such that they seal tightly around the extension tube 506 and secure the catheter 501 in position relative to the catheter dressing 500.

The circumferential seal member 530 is shown in the illustrated embodiment disposed adjacent a proximal end of the body 511. The seal member 530 may comprise the first or upper seal member 531 and the lower or second seal member 532. The seal members 531, 532 may be generally cylindrically “C” shaped. The upper seal member 531 is coupled to the displaceable member 515, and the lower seal member 532 is coupled to the body 511. This configuration allows the upper seal member 531 to be displaced away from the lower seal member 532 when the displaceable member 515 is in the open state, and to be coupled to the lower seal member 532 when the displaceable member 515 is the closed state. The seal member 530 can be formed from any suitable compliant material to facilitate forming a second circumferential seal around a portion of an extension tube 506 when the displaceable member is in the closed state. For example, the seal member 530 can be formed from silicone, polyurethane, thermoplastic elastomer, etc.

In the depicted embodiment of FIGS. 10B and 10C, when the displaceable member 515 is in the closed state, the first engagement member 522 is coupled to the second engagement member 524 such that the lower seal member 532 and the upper seal member 531 are compressed together. This configuration causes the circumferential seal member 530 to form an improved second circumferential seal around the extension tube 506 than in embodiments where the seal member 530 is not compressed. Additionally, the circumferential seal facilitates securement of the catheter 501 in position relative to the catheter dressing 500.

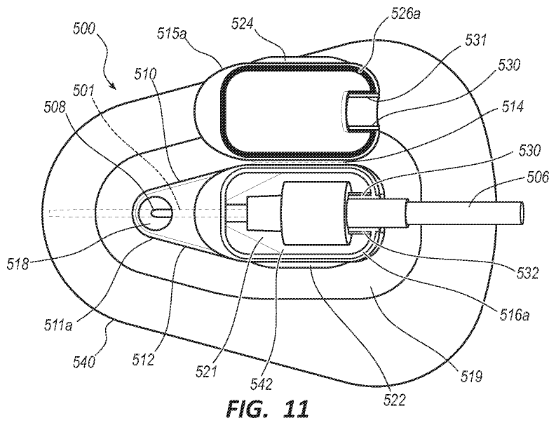

In the depicted embodiment, a first gasket 516 can extend circumferentially around the opening 521 and may be coupled to the body 511 and integral with the lower seal member 532 to facilitate sealing of the opening 521 by the displaceable member 515 and the first gasket 516 when the displaceable member 515 is in the closed state. In another embodiment, the first gasket 516 may not be integral with the lower seal member 532. In another embodiment of a catheter dressing 500a, as shown in FIG. 11, a second gasket 526a may be integral with a lower surface of the displaceable member 515a and configured to sealingly couple with a first gasket 516a when the displaceable member 515a is in the closed state. As depicted, the second gasket 526a is coupled to the upper seal member 531a. In some embodiments, the second gasket 526a may not be coupled to the upper seal member 531a. In another embodiment, the second gasket 526a may be configured to seal directly against the body 511a.

In some embodiments, the body 511 may include a first portion and a second portion. The first portion of the body 511 may be formed of a compliant material, such as polyurethane, thermoplastic elastomer, etc. The first portion of the body 511 may include a flange 519 configured to be coupled to the base plate 540 and a body portion extending upward from the flange 519. An upper edge of the body portion may form the first gasket 516. The second portion of the body 511 may be formed of any suitable rigid or semi-rigid thermoplastic material, such as polypropylene, polyethylene, polycarbonate, polyvinylchloride, polyurethane, etc. The second portion of the body 511 may be disposed over the upper portion of the first portion of the body 511 to substantially form an exoskeleton around the upper portion. The first portion and the second portion of the body 511 may be coupled using any suitable technique, such as over molding, two shot molding, etc. The material of the second portion of the body 511 can be more rigid than the material of the first portion of the body 511 such that the second portion of the body 511 provides adequate rigidity to support coupling of the body 511 with the displaceable member 515.

As depicted in the illustrated embodiment, the base plate 540 is coupled to the housing 510. The housing 510 may include the flange 519 to facilitate coupling of the base plate 540 to the housing 510 using any suitable technique, such as welding, gluing, bonding, etc. In the depicted embodiment, the base plate 540 includes an aperture 542 disposed in a middle portion of the base plate 540. The aperture 542 may be sized to permit passage of the base plate 540 over the inserted catheter 501 when the catheter dressing 500 is installed onto the patient. When installed, the base plate 540 may surround the catheter 501 at the insertion site 508 such that a first circumferential seal is formed around the catheter insertion site 508. The first circumferential seal may be in the plane of the base plate 540 and surround the catheter insertion site 508 a full 360 degrees. In other words, the first circumferential seal may surround the catheter insertion site 508 without breaks in the first circumferential seal such that microbial contamination of the catheter 501 and insertion site 508 may be prevented.

In the depicted embodiment, a sterile or sealed chamber 520 may be at least partially defined by the body 511, the displaceable member 515, the base plate 540, and the circumferential seal member 530. When the catheter dressing 500 is installed and the displaceable member is in the closed state, the sealed chamber 520 may be configured to provide a substantially sterile environment around the catheter 501 and insertion site 508. In other words, the environment of the sealed chamber 520 may be substantially free of microorganisms.

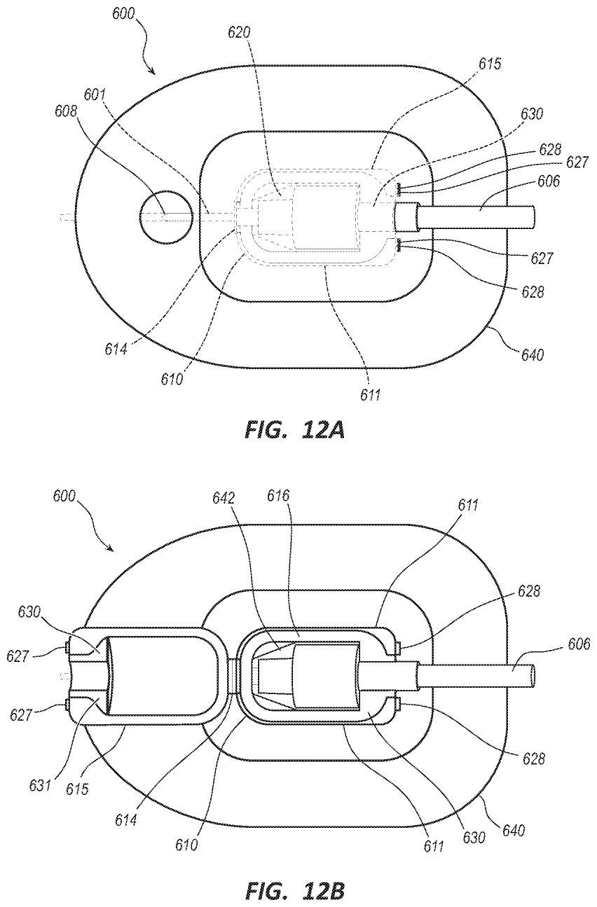

FIGS. 12A-12B depict an embodiment of a catheter dressing 600. In the illustrated embodiment, the catheter dressing 600 is composed of a housing 610 and a base plate 640. The housing 610 may be composed of a body 611, a displaceable member or lid 615, and a circumferential seal member 630.

In the illustrated embodiment, the body 611 includes a generally rectangular footprint. The body 611 may be formed of any suitable rigid or semi-rigid thermoplastic material. For example, the body 611 may be formed from polypropylene, polyethylene, polycarbonate, polyvinylchloride, etc., using any suitable forming technique, such as thermoforming, injection molding, etc.

In the depicted embodiment, the displaceable member 615 is configured to be displaced from an open state, as shown in FIG. 12B, to a closed state as shown in FIG. 12A. The displaceable member 615 may be sized to cover and seal an opening 621 of the body 611 when the displaceable member 615 is in the closed state. The displaceable member 615 can be distally displaced away from the opening 621 to permit access to an interior of the body 611 through the opening 621 when the displaceable member 615 is in the open state. A hinge 614 is shown to couple the displaceable member 615 to the body 611. The hinge 614 is disposed adjacent a distal end of the body 611 such that the displaceable member 615 is displaced distally relative to a longitudinal axis of the body 611. The hinge 614 may be a flexible hinge that allows for multiple actuations and provides a sealed coupling between the displaceable member 615 and the body 611.

The displaceable member 615 can include first engagement or clasp members 627 disposed adjacent a proximal end of the body 611 opposite of the hinge 614. The first engagement members 627 may be configured to engage with second engagement members 628 disposed adjacent a proximal end of the displaceable member 615 and securely couple the displaceable member 615 to the body 611 when the displaceable member 615 is in the closed state. The engagement members 627, 628 may include any suitable configuration capable of securing the displaceable member 615 and the body 611 together. For example, the engagement members 627, 628 may be frictional or snap fit joints, such as cantilever, annular, etc. In some embodiments, the engagement members 627, 628 can be fixedly coupled with the displaceable member 615 is in the closed state. In other embodiments, the engagement members 627, 628 can be releasably coupled when the displaceable member 615 is in the closed state. When in the closed state, the engagement members 627, 628 are configured to apply a compressive force to a first seal member 631 and a second seam member 632 such that they seal tightly around the extension tube 606 and secure the catheter 601 in position relative to the catheter dressing 600.

The circumferential seal member 630 is shown in the illustrated embodiment disposed adjacent a proximal end of the body 611. The seal member 630 may comprise a first or upper seal member 631 and a lower or second seal member 632. The seal members 631, 632 may be generally cylindrically “C” shaped. The upper seal member 631 may be coupled to the displaceable member 615, and the lower seal member 632 may be coupled to the body 611. This configuration allows the upper seal member 631 to be displaced away from the lower seal member 632 when the displaceable member 615 is in the open state and to be coupled to the lower seal member 632 when the displaceable member 615 is in the closed state. The seal member 630 can be formed from any suitable compliant material to facilitate forming a second circumferential seal around a portion of an extension tube 606 when the displaceable member 615 is in the closed state.

In the depicted embodiment of FIG. 12A, when the displaceable member 615 is in the closed state, the first engagement members 627 are coupled to the second engagement members 628 such that the lower seal member 632 and the upper seal member 631 are compressed together. This configuration causes the circumferential seal member 630 to form an improved second circumferential seal around the extension tube 606 than in embodiments where the circumferential seal member 630 is not compressed. Additionally, the circumferential seal facilitates securement of the catheter 601 in position relative to the catheter dressing 600.

In the depicted embodiment, a gasket 616 extends circumferentially around the opening 621 and is coupled to the body 611 and to the lower seal member 632 to facilitate sealing of the opening 621 by the displaceable member 615 and the gasket 616 when the displaceable member 615 is in the closed state. In another embodiment, the gasket 616 may not be coupled to the lower seal member 632. In some embodiments, a second gasket 616 may be coupled to a lower surface of the displaceable member 615 and configured to sealingly couple with the gasket 616 when the displaceable member 615 is in the closed state.

As depicted in the illustrated embodiment, the base plate 640 is coupled to the housing 610. The housing 610 may include a flange 619 to facilitate coupling of the base plate 640 to the housing 610. In the depicted embodiment, the base plate 640 includes an aperture 642 disposed in a middle portion of the base plate 640. The aperture 642 may be sized to permit passage of the base plate 640 over an inserted catheter 601 and the extension tube 606 when the catheter dressing 600 is installed onto the patient. An optional transparent viewing window 618 may be disposed adjacent the distal portion such that a catheter 601 and catheter insertion site 608 may be viewed by a clinician. When installed, the base plate 640 may surround the catheter 601 at the insertion site 608 such that a first circumferential seal is formed around the catheter insertion site 608. The first circumferential seal may be in the plane of the base plate 640 and surround the catheter insertion site 608 a full 360 degrees. In other words, the first circumferential seal may surround the catheter insertion site 608 without breaks in the first circumferential seal such that microbial contamination of the catheter 601 and insertion site 608 may be prevented.

In the depicted embodiment, a sterile or sealed chamber 620 may be at least partially defined by the body 611, the displaceable member 615, the base plate 640, and the circumferential seal member 630. When the catheter dressing 600 is installed and the displaceable member 615 is in the closed state, the sealed chamber 620 may be configured to provide a substantially sterile environment around the catheter 601 and insertion site 608. In other words, the environment of the sealed chamber 620 may be substantially free of microorganisms.

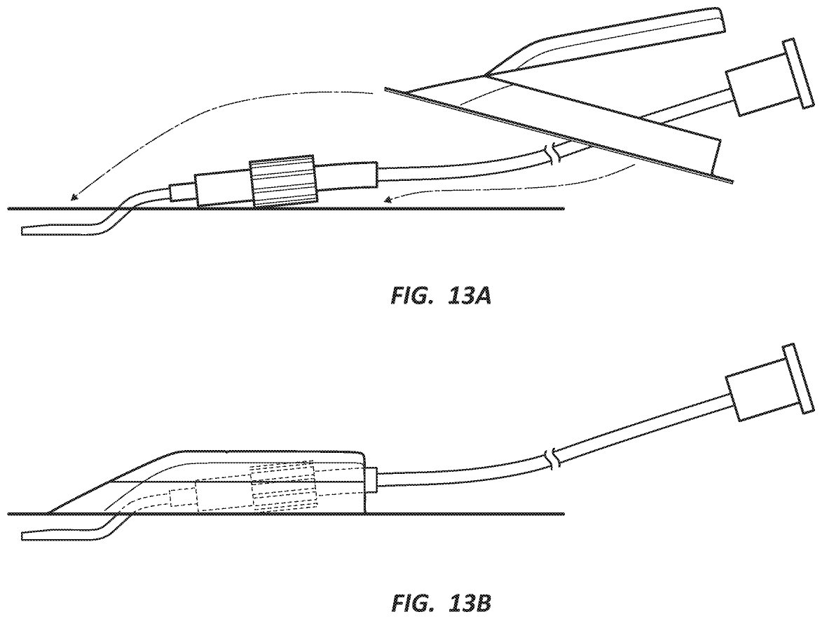

In use, the catheter dressing of any one of the catheter dressing embodiments disclosed herein or any catheter dressing within the scope of this disclosure, as exemplarily illustrated in FIGS. 13A and 13B, may be disposed over a proximal portion of an extension tubing assembly in a ready state. The extension tubing assembly may be composed of a distal connector, a proximal connector, and an extension tube. In a ready or pre-deployed state, the extension tube may extend through an aperture of a base plate and through an opening of a body of the catheter dressing. A displaceable member may be displaced away from the body in an open state. A seal member may be in a non-sealing state, where an upper seal member is displaced away from a lower seal member. The extension tubing assembly may be flushed with a fluid (e.g., saline or heparinized saline) to remove air from the extension tubing assembly. A catheter (e.g., IV catheter, PIC catheter, central venous catheter, etc.) may be inserted into a patient by a clinician. The distal connector may be sealingly connected to a hub of the catheter to form a fluid communication between the catheter and the extension tube assembly.

The catheter dressing may be translated distally along the extension tube until it reaches a pre-installed state, where the catheter and distal connector have passed through an aperture of a base plate and are disposed within the body and the base plate is disposed adjacent the patient's skin. In certain instances, the extension tube assembly may be pressed into and coupled with a lower seal member to facilitate stabilization of the catheter and the extension tubing assembly prior to further manipulation of the catheter dressing.

In an installed state, as depicted by FIG. 13B, a lid or displaceable member may be displaced toward and secured to the body to close, lock, and seal an opening of the body. An upper seal member and the lower seal member can form a circumferential seal around a portion of the extension tube assembly. The base plate may be adhered to the patient's skin by removal of a release liner to form a first circumferential seal around the catheter at a catheter insertion site. A sealed or sterile chamber may be defined by the base plate, the body, the displaceable member, the first circumferential seal, and the second circumferential seal.

Any one of the catheter dressings previously described may be manufactured, packaged, and sterilized prior to delivery to a healthcare worker and used as described above. The catheter dressing may be packaged individually or as a system or kit. The system can include the manufactured catheter dressing and an extension tube assembly. The catheter dressing may be customizable in size for use in pediatric or adult applications where a size of a catheter may vary according to the application. Additionally, a size (e.g., inner diameter) of a seal member of the catheter dressing may be customizable to ensure a circumferential seal around the extension tube assembly. The extension tube assembly may include different configurations customized for different applications, for example pediatric and adult. The extension tube assembly can include a distal connector, a proximal connector, and a tube, all of which can be customizable in size and/or flexibility dependent upon the application of use.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A catheter dressing, comprising:
a base plate configured to form a first circumferential seal around a catheter;
a housing coupled to the base plate, the housing comprising:
a body, and
a displaceable member;
a circumferential seal member configured to form a second circumferential seal around the catheter; and
a sealed chamber within the housing disposed between the first and second circumferential seals.

2. The catheter dressing of claim 1, wherein the base plate comprises:
a film layer; and
an aperture disposed in the film layer;
wherein the catheter is disposed through the aperture when the catheter dressing is installed on a patient.

3. The catheter dressing of claim 2, wherein the film layer comprises any one of a hydrocolloid and a polyurethane film.

4. The catheter dressing of claim 2, wherein the film layer comprises an adhesive.

5. The catheter dressing of claim 2, wherein the base plate further comprises a clear viewing window.

6. The catheter dressing of claim 1, wherein the body comprises one of a rigid or semi-rigid plastic material, an elastomeric material, and any combination thereof.

7. The catheter dressing of claim 1, wherein the displaceable member and the circumferential seal member comprise a unibody construction.

8. The catheter dressing of claim 1, wherein the displaceable member is configured to close an opening of the body when the displaceable member is in a closed state.

9. The catheter dressing of claim 1, wherein the displaceable member is configured to non-releasably couple to the body.

10. The catheter dressing of claim 1, wherein the displaceable member is configured to releasably couple to the body.

11. The catheter dressing of claim 1, wherein the housing further comprises:
a hinge disposed between the displaceable member and the body; and
a sealing gasket configured to form a seal between the body and the displaceable member when the displaceable member is in a closed state.

12. The catheter dressing of claim 1, wherein the catheter dressing is slidingly disposed over an extension tube when the displaceable member is in an open state.

13. The catheter dressing of claim 1, wherein the circumferential seal member comprises:
a first seal member coupled to the displaceable member; and
a second seal member coupled to the body;
wherein the first seal member is configured to couple with the second seal member to form the second circumferential seal around the catheter when the circumferential seal member is in a sealing state.

14. The catheter dressing of claim 13, wherein the second seal member comprises a larger height than the first seal member, and wherein the second seal member is configured to stabilize the catheter.

15. The catheter dressing of claim 13, wherein the first seal member is disposed away from the second seal member when the circumferential seal member is in a non-sealing state, and wherein the first seal member is sealingly coupled to the second seal member when the circumferential seal member is in the sealing state.

16. The catheter dressing of claim 1, wherein the housing comprises at least one locking member, comprising:
at least one first engagement member coupled to the body; and
at least one second engagement member coupled to the displacement member.

17. The catheter dressing of claim 16, wherein the at least one first and second engagement members are configured to lock the displaceable member to the body in a closed state such that the circumferential seal member is radially compressed.

18. The catheter dressing of claim 1, further comprising a conformable member coupled to the displaceable member and configured to conform around and stabilize the catheter when the displaceable member is in a closed state.

19. The catheter dressing of claim 18, wherein the conformable member comprises an antimicrobial substance.

20. The catheter dressing of claim 1, wherein the body comprises a clear viewing window.

21. The catheter dressing of claim 1, wherein the sealed chamber is at least partially defined by the base plate, the body, the displaceable member, and the circumferential seal member.

22. The catheter dressing of claim 1, wherein the circumferential seal member comprises:
a first seal member coupled to a first seal member support, where the first seal member support is coupled to the displaceable member; and
a second seal member coupled to a second seal member support, where the second seal member support is coupled to the body;
wherein the first seal member support is configured to fixedly couple with the second seal member support when the circumferential seal member is in a sealing state; and
wherein the first seal member is configured to couple with the second seal member to form the second circumferential seal around the catheter when the circumferential seal member is in the sealing state.

23. The catheter dressing of claim 22, further comprising a gasket coupled to at least one of the displaceable member, the base plate, the body, the first seal member support, and the second seal member support.

24. The catheter dressing of claim 23, wherein the body defines an opening configured to receive an extension tube when the displaceable member is in an open state and to surround the extension tube when the displaceable member is in a closed state.

25. The catheter dressing of claim 22, further comprising:
a first securement strap coupled to the first seal member support; and
a second securement strap coupled to the second seal member support;
wherein the first securement strap and the second securement strap are configured to stabilize the catheter dressing to a patient.

26. The catheter dressing of claim 1, wherein the circumferential seal member is configured to be radial outwardly distendable when in a ready state and to be elastically radial inwardly retractable when in a sealing state.

27. The catheter dressing of claim 1, wherein the catheter is any one of a straight peripheral intravenous (IV) catheter, an integrated peripheral IV catheter, a peripherally inserted central (PIC) catheter, a midline catheter, a central line catheter, and an umbilical catheter.

28. A catheter dressing system, comprising:
a catheter dressing, comprising:
a base plate configured to form a first circumferential seal around a catheter;
a housing coupled to the base plate, the housing comprising:
a body, and
a displaceable member;
a circumferential seal member configured to form a second circumferential seal around the catheter; and
a sealed chamber within the housing disposed between the first and second circumferential seals; and
an extension tube assembly, comprising:
an extension tube; and
a connector disposed at an end of the extension tube.

29. The catheter dressing system of claim 28, wherein the catheter dressing is disposed around the extension tube when the catheter dressing system is in a ready state, and wherein the catheter dressing is configured to be distally displaced along the extension tube when the catheter dressing system is in an pre-installed state.

30. The catheter dressing system of claim 28, wherein the connector is configured to be coupled to a hub of an intravenous (IV) catheter and configured to be retained from proximal displacement by the circumferential seal member.

31. The catheter dressing system of claim 30, wherein the circumferential seal member sealingly couples to the extension tube to form the second circumferential seal around the connector.

32. A method of maintaining sterility of an indwelling catheter, comprising:
obtaining a catheter dressing;
displacing the catheter dressing along an extension tube and over a catheter; and
forming a sealed chamber within the housing.

33. The method of claim 32, wherein forming the sealed chamber comprises:
closing a displaceable member to form a seal between the displaceable member and a body of the catheter dressing;
closing a circumferential seal member to form a second circumferential seal around the extension tube; and
securing a base plate to skin adjacent a catheter insertion site of a patient to form a first circumferential seal around the catheter.

34. The method of claim 33, further comprising removing the catheter dressing from a patient, wherein removing the catheter dressing from the patient comprises:
displacing the displaceable member to an open state;
releasing the base plate from skin of the patient; and
displacing the catheter dressing proximally over the catheter and the extension tube.

35. The method of claim 33, further comprising stabilizing the catheter by coupling the extension tube with a lower seal member of the circumferential seal member.

36. The method of claim 33, further comprising adding an active agent to a conformable member of the catheter dressing.

37. The method of claim 36, wherein the active agent is an antimicrobial substance.

38. A method of maintaining sterility of an indwelling catheter, comprising:
obtaining a combination catheter dressing, comprising
a catheter dressing; and
an extension tubing assembly comprising:
an extension tube; and
a connector coupled to a distal end of the extension tube;
wherein the catheter dressing is circumferentially pre-deployed around the extension tube;
flushing the extension tubing assembly with a fluid;
coupling the connector of the flushed extension tubing assembly to a hub of an inserted catheter;
sliding the catheter dressing distally over the extension tube and into position around the inserted catheter and the connector;
pressing the extension tube into a base of a housing of the catheter dressing to pre-stabilize the inserted catheter and connector;
closing a lid of the catheter dressing into a locked and a sealing position; and
peeling off an adhesive backing of a base plate of the catheter dressing; and
coupling the catheter dressing and the inserted catheter to the skin of a patient.
